(19) United States
(12) Patent Application Publication
(10) Pub. No.: US 2023/0320641 A1
(43) Pub. Date: Oct. 12, 2023

(54) SYSTEMS AND METHODS TO FACILITATE SELF-ANALYSIS THROUGH DATA BASED ON INPUTS ASSOCIATED WITH PSYCHOLOGICAL STATES OF A USER

(71) Applicants: Thelma Alida Van der Werff, Warkworth RD 2 (NZ); Mary Ashby-Green, Orewa (NZ); Abhay Mohan Wadhwa, New York, NY (US); Anoop Kumar Kushwaha, Lucknow (IN)

(72) Inventors: Thelma Alida Van der Werff, Warkworth RD 2 (NZ); Mary Ashby-Green, Orewa (NZ); Abhay Mohan Wadhwa, New York, NY (US); Anoop Kumar Kushwaha, Lucknow (IN)

(21) Appl. No.: 17/719,325

(22) Filed: Apr. 12, 2022

(51) Int. Cl. A61B 5/16; G16H 40/67; A61B 5/00

(52) U.S. Cl. CPC A61B 5/165; G16H 40/67; A61B 5/7475

(57) ABSTRACT

Disclosed herein is a method to facilitate self-analysis through data based on inputs associated with psychological states of a user. Accordingly, the method may include receiving a request for maintaining a mental health of the user from a user device associated with the user, transmitting colors and a first instruction to the user device, receiving a first response of the user associated with the colors from the user device, analyzing the first response and the first instruction of the user using an algorithm, determining psychological state of the user associated with the selection of the colors, identifying color for the maintaining of the mental health of the user, identifying a product of the color for the user, generating a notification of the product, transmitting the notification to the user device, storing the psychological state of the user associated with the selection of the colors and the list of colors.

### FIELD OF THE INVENTION

Generally, the present disclosure relates to the field of data processing. More specifically, the present disclosure relates to methods and systems to facilitate self-analysis through data based on inputs associated with psychological states of a user.

### BACKGROUND OF THE INVENTION

The field of data processing is technologically important to several industries, business organizations, and/or individuals. In particular, the use of data processing is prevalent for facilitating self-analysis through data based on inputs associated with psychological states of a user.

Given that the ongoing technological advancements, the methods for mental health treatments are increasing day by day. Existing techniques to reprocess emotional conflicts and personal life conditions are deficient with regard to several aspects. For instance, current technologies use either an NLP technique or a Color Therapy in conjunction with the physical aspects of color. Further, current technologies use color therapy with respect to the chakras in the human body, etc. Furthermore, current technologies are generalized and the process is the same for everyone using the system. Further, current technologies become overwhelming overtime for the user. Current technologies further are generalized and the same treatment is meant to be used on each individual using the therapy/treatment. Furthermore, current technologies do not use color psychology to access the subconscious mind and thus balance the negative conditions. Moreover, current technologies do not provide clear insight and answers to an individual so that a positive change can take place.

Therefore, there is a need for improved methods and systems to facilitate self-analysis through data based on inputs associated with psychological states of a user that may overcome one or more of the above-mentioned problems and/or limitations.

### SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

Disclosed herein is a method to facilitate self-analysis through data based on inputs associated with psychological states of a user, in accordance with some embodiments. Accordingly, the method may include receiving, using a communication device, a request for maintaining a mental health of the user from at least one user device associated with the user. Further, the method may include transmitting, using the communication device, a plurality of colors and at least one first instruction to the at least one user device based on the request. Further, the at least one user device may include at least one presentation device. Further, the at least one presentation device may be configured for presenting the at least one instruction and the plurality of colors to the user. Further, the method may include receiving, using the communication device, a first response of the user associated with the plurality of colors from the at least one user device. Further, the first response may include a selection of at least one of the plurality of colors and a first reaction of the user associated with the selection of the at least one of the plurality of colors. Further, the method may include analyzing, using a processing device, the first response and the at least one first instruction of the user using at least one algorithm. Further, the method may include determining, using the processing device, a psychological state of the user associated with the selection of the at least one of the plurality of colors based on the analyzing of the first response and the at least one first instruction. Further, the method may include identifying, using the processing device, at least one color for the maintaining of the mental health of the user based on the determining. Further, the method may include identifying, using the processing device, at least one product of the at least one color for the user based on the identifying of the at least one color. Further, the method may include generating, using the processing device, a notification of the at least one product based on the identifying of the at least one product. Further, the method may include transmitting, using the communication device, the notification to the at least one user device. Further, the method may include storing, using a storage device, the psychological state of the user associated with the selection of the at least one of the plurality of colors and the selection of the at least one of the set of colors.

Further disclosed herein is a system to facilitate self-analysis through data based on inputs associated with psychological states of a user, in accordance with some embodiments. Accordingly, the system may include a communication device configured for receiving a request for maintaining a mental health of the user from at least one user device associated with the user. Further, the communication device may be configured for transmitting a plurality of colors and at least one first instruction to the at least one user device based on the request. Further, the at least one user device may include at least one presentation device. Further, the at least one presentation device may be configured for presenting the at least one instruction and the plurality of colors to the user. Further, the communication device may be configured for receiving a first response of the user associated with the plurality of colors from the at least one user device. Further, the first response may include a selection of at least one of the plurality of colors and a first reaction of the user associated with the selection of the at least one of the plurality of colors. Further, the communication device may be configured for transmitting a notification to the at least one user device. Further, the system may include a processing device communicatively coupled with the communication device. Further, the processing device may be configured for analyzing the first response and the at least one first instruction of the user using at least one algorithm. Further, the processing device may be configured for determining a psychological state of the user associated with the selection of the at least one of the plurality of colors based on the analyzing of the first response and the at least one first instruction. Further, the processing device may be configured for identifying at least one color for the maintaining of the mental health of the user based on the determining. Further, the processing device may be configured for identifying at least one product of the at least one color for the user based on the identifying of the at least one color. Further, the processing device may be configured for generating the notification of the at least one product based on the identifying of the at least one product. Further, the system may include a storage device communicatively coupled with the processing device. Further, the storage device may be configured for storing the psychological state of the user associated with the selection of the at least one of the plurality of colors and the selection of the at least one of the list of colors.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform consistent with various embodiments of the present disclosure.

FIG. 2 is a flow diagram of a method to facilitate self-analysis through data based on inputs associated with psychological states of a user, in accordance with some embodiments.

FIG. 3 is a flow diagram of a method to facilitate self-analysis through data based on inputs associated with psychological states of a user, in accordance with some embodiments.

FIG. 4 is a continuation flow diagram of FIG. 3.

FIG. 5 is a flow diagram of a method to facilitate self-analysis through data based on inputs associated with psychological states of a user, in accordance with some embodiments.

FIG. 6 is a flow diagram of a method to facilitate self-analysis through data based on inputs associated with psychological states of a user, in accordance with some embodiments.

FIG. 7 is a flow diagram of a method to facilitate self-analysis through data based on inputs associated with psychological states of a user, in accordance with some embodiments.

FIG. 8 is a flow diagram of a method to facilitate self-analysis through data based on inputs associated with psychological states of a user, in accordance with some embodiments.

FIG. 9 is a flow diagram of a method to facilitate self-analysis through data based on inputs associated with psychological states of a user, in accordance with some embodiments.

FIG. 10 is a flow diagram of a method to facilitate self-analysis through data based on inputs associated with psychological states of a user, in accordance with some embodiments.

FIG. 11 is a flow diagram of a method to facilitate self-analysis through data based on inputs associated with psychological states of a user, in accordance with some embodiments.

FIG. 12 is a flow chart of a method to facilitate self-analysis through data based on inputs associated with psychological states of a user, in accordance with some embodiments.

FIG. 13 is a flow chart of a method to facilitate self-analysis through data based on inputs associated with psychological states of a user, in accordance with some embodiments.

FIG. 14 is a flow chart of a method to facilitate self-analysis through data based on inputs associated with psychological states of a user, in accordance with some embodiments.

FIG. 15 is a flow chart of a method to facilitate self-analysis through data based on inputs associated with psychological states of a user, in accordance with some embodiments.

FIG. 16 is a flow chart of a method to facilitate self-analysis through data based on inputs associated with psychological states of a user, in accordance with some embodiments.

FIG. 17 is a flow chart of a method to facilitate self-analysis through data based on inputs associated with psychological states of a user, in accordance with some embodiments.

FIG. 18 is a block diagram of a system to facilitate self-analysis through data based on inputs associated with psychological states of a user, in accordance with some embodiments.

FIG. 19 is a block diagram of the system to facilitate self-analysis through data based on inputs associated with psychological states of a user, in accordance with some embodiments.

FIG. 20 is a block diagram of a computing device for implementing the methods disclosed herein, in accordance with some embodiments.

### DETAIL DESCRIPTIONS OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of methods and systems to facilitate self-analysis through data based on inputs associated with psychological states of a user, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor and at least one actuator. Examples of the one or more client devices and/or the server computer may include, a desktop computer, a laptop computer, a tablet computer, a personal digital assistant, a portable electronic device, a wearable computer, a smart phone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice based interface, gesture based interface etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third party database, public database, a private database and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role based access control, and so on.

Further, one or more steps of the method disclosed herein may be initiated, maintained, controlled and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g. username, password, passphrase, PIN, secret question, secret answer etc.) and/or possession of a machine readable secret data (e.g. encryption key, decryption key, bar codes, etc.) and/or or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera and so on. Likewise, the one or more steps may include receiving, using the communication device, the one or more embodied characteristics from one or more biometric sensors.

Further, one or more steps of the method may be automatically initiated, maintained and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device etc.) corresponding to the performance of the one or more steps, physical state and/or physiological state and/or psychological state of the user, and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor etc.), a biometric sensor (e.g. a fingerprint sensor), etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data and any intermediate data therebetween corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

Overview:

The present disclosure describes methods and systems to facilitate self-analysis through data based on inputs associated with psychological states of a user. Further, the data is derived from algorithms. Further, the present disclosure describes methods, systems, apparatuses, and devices for facilitating recommending specific colored products based on a psychology of a user. Further, the disclosed system may be configured for recommending color-favored products based on a psychological state of the user (or users). Further, as every individual is different and no single human evolves with the same mental state, the disclosed system may use features of NLP and Color Psychology to access the subconscious mind and thus balance the negative conditions, getting the user to leave behind long-lasting patterns, get clear insight and answers so that a positive change can take place.

Further, the disclosed system may be associated with a software platform such as a mobile application. Further, the mobile application may be a unique combination of Neuro-Linguistic Programming (NLP) with the psychology of color to access and change the subconscious negative state of mind processed by a programmable code that is executed by one or more processors, in which a user-friendly graphical interface showing a series of the interactive communication process and their evaluation for a method which help in mental health wellness.

Further, the disclosed system may recognize user input at all stages for mapping their color patterns over time vs their positive & negative state of mind which are further analyzed via an app algorithm to assist & notify the user for maintaining their mental health via color adoption in their daily life. Further, one or more of processors associated with the disclosed system may be programmed to receive the user's input while exerting their response against a log of use dataset, generate evaluations against those inputs, and visually indicate them via algorithmically derived guided statements. Further, the processors may be programmed to articulate their concerns or issues, identify the barriers that prevent them from overcoming their issues, discover their solutions, visualize how they would feel if their problems were solved, and empower them to get rid of those ailments and find their new perspective.

Further, the disclosed system may be designed to show users new pathways, possibilities, and options so they can transform their life and eliminate negative thoughts.

Further, the disclosed system may be associated with a unique self-empowerment tool that combines Neuro-Linguistic Programming (NLP) and color psychology for finding the most preferred color and least preferred color for the user, enabling the user to implement their most preferred color in form of clothing or accessory for better integration of the positive subconscious thoughts, and also for an easy and quick option of carrying their positive color and thus closure with them, via finding apparel and accessories of the evaluated color from the app algorithm. This methodology uses color to allow the user to gain a deeper insight into life through their color choices. This process is especially suited for those who wish to develop themselves, get clarity, and wish to fully realize their potential. Further, the disclosed system may be configured for prompting individuals to recapitulate the healing experience to give the user a long-lasting impression of the unique closure, that is generated by user pattern maps.

Further, the disclosed system may be configured for performing analysis of a procedure that the user has done through various algorithms. Further, a cartesian analysis & color body map along with color history mapping and calendar tracking from the same is generated by the disclosed system for the user to review. Further, the user may know their choice of color along with the inputs they chose and the intensity of its impact mapped on a single graphical presentation. All the data may be stored with a matrix of time, that may be mapped and presented to the user where an in-depth analysis of the user's progress may be tracked and assisted to reach their desired goal. The user with the adoption of their positive color in their daily life through the clothes and accessories they wear (via user input), and where they feel that particular color (a color that generates positive thoughts in users mind) on their body is suggested by the disclosed system.

Further, the disclosed system combines the psychology of color and Neuro-Linguistic Programming (NLP). The language of color helps one understand their issues on a conscious level and gives them insight into themself and what is blocking them from moving forward. This procedure gives one an understanding of themself through their color choices. The disclosed system may access and change the subconscious negative state of mind. Further, the disclosed system may be configured for personal transformation via a self-help procedure using the psychology of color and Neuro-Linguistic Programming to restore mental wellness and stability by allowing both men and women with mental conflicts to receive a therapy based on the psychology of color and Neuro-Linguistic Programming via a mobile app and to be treated.

Further, the mobile application may be a self-help tool for personal transformation or mind-body harmony. The language of color helps user unravel their issues and resolve them. Further, the disclosed system may be configured self-healing using the psychology of color and Neuro-Linguistic Programming (NLP) on a display interface via the mobile application.

Further, the disclosed method includes steps of sequentially displaying a list of colors including a plurality of colors by which a user can select those colors along with predefined statements which indicate the intensity of the conditions; storing preference color information and all user-driven inputs throughout the process for a selected color and a selection order selected by the user from the color displayed by the terminal/user interface; extracting all the information corresponding to the preferred color information from the terminal along with all user-driven inputs throughout the process, and displaying the outputs by the terminal. Once user adapts to the mobile application, the user may change their mindset via their perspective and their inner dialogue and step into a new positive perspective and what may follow are new feelings, thoughts, perspectives, attitudes, and actions. The mobile application (or app) analyses the user pattern for the clothes they wear (via user input), and where they feel their positive colors on their body, suggests the user with the adoption of their positive colors in their daily life. The mobile application consists of different numbers of steps that lead towards the results of identifying various issues which are in the user's subconscious mind and help one to understand how they think and feel and how they process their experiences. It provides techniques to interrupt users' current thought patterns and examine what's going on internally helping them break away from old patterns to make changes within.

Further, in an embodiment, in the disclosed method, at first, the user has to register themselves by filling up the signup or login form. Based on these data, user profiles will be created for all the users. Further, the user may access any methods within the given options (four options), which are—A. Colornostics process, B. Mindset of the Day, C. Quick fix Problem Solver, and D. Your Color Analysis. Each method has its evaluation system via several logics, databases, and architecture but all of these are centrally connected to the main database. The colornostics process may be a five-step process, where the users may go through a series of sequential steps which starts from—identifying their mental condition and finding the root cause behind those issues which acts as a blockage in their progress via psychology of color and Neuro-Linguistic Programming (NLP). After receiving users' responses, the app algorithm starts a healing procedure, which starts from the transition and slowly prompts the user to recapitulate the healing experience.

Further, the colornostics process may be a 5-step process. Further, for a first color (Card 1), the users may be asked to think about the problem that is going on in their mind without trying to solve it or explain it, while looking at the 20 colors displayed on the screen, intuitively select a color which they dislike the most at that particular moment. Further, the users may exert their response intuitively by choosing the color which they dislike the most at that particular moment. Further, the users may be asked to choose one or more statements that reflect their current state of mind (what they often think to themselves), along with the intensity of the statement on a scale of mild (ss=1), medium (ss=2), and strong (ss=3). The response may be fetched by the app algorithm and stored in the memory database for future usage. Further, for card 2, the users may be asked to think about the probable reasons for their issues which are blocking them psychologically while looking at 20 colors displayed on a screen. Further, the users may have to pick their least favorite color at that particular moment. Further, the color that the users chose previously in Card 1 is not available in the selection options. Further, for a response for the card 2, the users may exert their response intuitively by choosing the color which the user may dislike the most at that particular moment for the Card. Further, the user may be asked to choose one or more statements that represent their current state of mind, along with the intensity of the statement on a scale of mild (ss=1), medium (ss=2), and strong (ss=3). Further, the response for the card 2 may be fetched by the app algorithm and stored in a memory database for future usage. Further, the colornostics process may include preparation. Further, in the display interface, algorithmically derived guided messages are shown which guides the user to optimize the treatment procedure. In this part, the user may sit comfortably, take a breath, and feel relaxed in front of their device screens. Further, the colornostics process may include a transition state. Further, in the transition state, algorithmically derived messages are guide users, where users have to release all those mental ailments which block them to maintain a healthy mental state and prepared to be mentally free themselves from those issues. In this transition state (or period), an audiovisual clip plays on the device screen, where the color transition takes place with respective audio backgrounds that may enhance the treatment procedure. Further, the colornostics process may include a response for transition. Further, five options may be given for the user to spontaneously select the transition modes from waterfall, beach, mountain top, forest, and fire. Further, the user may select the duration of the transition. Further, colors that may be selected in the previous stages as Card 1 and Card 2 are seen here vanishing with the transition mode selected by the user in the 30 seconds/60-second video. Further, the colornostics process may be associated with a color Summary. Further, in the display interface, the color summary may be displayed. Further, the users may see the summary of the colors that the user may choose for Card 1 & Card 2. After completing the transition period, the users may be asked to think about their state of mind if their issue is resolved and the user is free of their issues, while looking at 20 (here the color that they chose previously in Cards 1, 2 is not available in the selection options) colors displayed on the screen, instinctually select a color (card 3) which they like the most at that particular moment. Further, for a response for the card 3, the users may exert their response intuitively by choosing a color that they like the most for Card 3 at that particular moment. While selecting the color, the user may select the one or more statements that represent their current state of mind along with the intensity of the statement on a scale of mild (ss=1), medium (ss=2), and strong (ss=3). The response is fetched by the app algorithm and stored in the memory database for future usage. Further, for card 4, the users may be asked to think about their newly developed mindset that has been activated in their new perspective, while looking at 20 colors (here the color that they chose previously in Cards 1, 2, 3 is not available in the selection options) displayed on the screen, intuitively select a color (Card 4) which they like the most at that particular moment. Further, for a response for Card 4, the users may exert their response intuitively by choosing a color that they like the most for Card 4 at that particular moment and the response is fetched by the app algorithm and stored in the memory database for future usage. While selecting the most favorite color, the user may have to select one or more statements that represent their current state of mind along with the intensity of the statement on a scale of mild (ss=1), medium (ss=2), and strong (ss=3). The response is fetched by the app algorithm and stored in the memory database for future usage. Further, for card 5, the users may be asked to think about their ongoing growth and mental sanity, while looking at 20 colors (here the color that they chose previously in Cards 1, 2, 3, 4 is not available in the selection options) displayed on the screen, intuitively select a color (Card 5) which they like the most at that particular moment. Further, for a response for Card 5, the users may exert their response intuitively by choosing a color which they like the most for Card 5 at that time. While selecting the color they have to select one or more statements that represent their current state of mind along with the intensity of the statement on a scale of mild (ss=1), medium (ss=2), and strong (ss=3). The response is fetched by the app algorithm and stored in the memory database for future usage. Further, the colornostics process may be associated with a color summary for 3, 4, 5. Further, in the display interface, the color summary may be displayed. Further, the users may see the summary of the colors that they chose for Card 3, Card 4 & Card 5. Further, the users may see the colors that the user chose for Card 3. Further, the user may be asked to choose a body part shown in the display interface where they feel that color which they choose for completing the procedure of color body mapping. Further, for a response for Color Body Map, the users may exert their response intuitively by choosing the body parts and the response is shown through the Color Body Map. Further, the users can see the colors that they chose for Card 4, and similarly the user may be asked to choose a body part shown in the display interface where the user may feel that color for completing the color body mapping. Further, for a response for the Color Body Map, the users may exert their response intuitively by choosing the body parts and the response is shown through the Color Body Map. Further, the users may see the colors that they chose for Card 5 and the user may be asked to choose a body part shown in the display interface where they feel that color for completing the color body mapping. Further, for a response for the Color body map: The users exert their response intuitively by choosing the body parts and the response is shown through the Color Body Map. After going through this whole procedure where users are asked to select a body location where they feel the colors in Card 3, Card 4, and Card 5. The location on the body may be a head area, chest area, stomach area, thighs area, legs area, hands area. The users may exert their response and the summary for the same is displayed simultaneously. The response is fetched by the app algorithm and stored in the memory database for future usage. Further, the disclosed system may be associated with a Summary. Further, algorithmically derived messages may be shown in this section which indicates the completion of the procedure. Further, the analysis of the previous selection that the user has done may be carried out through the App Algorithm (CAA). Further, the user may see their choice of color along with the statements they chose and the intensity of its impact mapped on a single graph. This is shown for Cards 3, 4 and 5, this is stored with a value of time, which can be further mapped and presented to the user.

Further, the disclosed method may be associated with a mindset of the day process that may be a one-step process. Further, for color card 1, first color (Card 1) users may be asked to think about a mindset that they want for the day, while looking at the 20 colors displayed on the screen, intuitively select a color. Further, for a response for Card 1, the users may exert their response intuitively by choosing a color that represents their mindset for the day. Further, for the color Summary for Card 1, the selected color may be displayed here in the section of the color summary. Further, algorithmically derived messages may be shown in this section and users may be asked to select one or more statements that represent their current state of mind along with the intensity of the statement on a scale of mild (ss=1), medium (ss=2), and strong (ss=3). The users may exert their response by selecting these statements which described their new thought, and the response is fetched by the app algorithm and stored in the memory database for future usage. Further, the users may be asked to choose a body part shown in the display where they want to feel the previous color. Further, for a response for Color Body Map, the users may exert their response intuitively by choosing a body part that described their current state of mind and the response is shown through the Color Body Map. In the display interface, the color summary may be displayed. Users can see the colors that they chose for the card 1 which represents their new thought. The analysis of the previous selection that the user has done may be carried out through Colornostics App Algorithm (CAA). Further, the user may see their choice of color along with the statements they chose and the intensity of its impact mapped on a single graph. This is shown for Card 1.

Further, the disclosed method may be associated with quick-fix problem solver process that may be a two-step process. Further, for the first color (Card 1), the users may be asked to think about the trouble that is ongoing in their mind without trying to solve it or explain it, while looking at the 20 colors displayed on the screen, intuitively select a color which they dislike the most at that particular moment. Further, for a response for Card 1, the users may exert their response intuitively by choosing a color that they dislike the most at that particular moment. Further, the selected color (or color) may be displayed here as a color summary. Further, algorithmically derived messages may be shown in this section and the users may be asked to select one or more statements from the given list which is relatable most deeply with their ongoing trouble along with the intensity of the statement on a scale of mild (ss=1), medium (ss=2), and strong (ss=3). The users exert their response by selecting the statements that represent their current state of mind, and the response is fetched by the app algorithm and stored in the memory database for future usage. Further, for card 2, the users may be asked to think about their state of mind if their issue is resolved and the users may be free of their issues while looking at the colors in the display. Further, the users may be asked to choose a color that represents their new experience. Further, for a response for Card 2, the users exert their response intuitively by choosing a color that they think is best suitable for representing their new experience at that particular moment. Further, the color (or selected color) may be displayed here as a color summary. Further, algorithmically derived messages may be shown in this section and the users are asked to select one statement from the given list which is relatable with their current state along with the intensity of the statement on a scale of mild (ss=1), medium (ss=2), and strong (ss=3). The users exert their response by selecting the statements that represent their current state of mind, and the response is fetched by the app algorithm and stored in the memory database for future usage. This statement shows how they feel if the issue was solved. Further, in the display interface, the color summary may be displayed. Further, the users may see the colors that they chose for Card 1 & Card 2 they select previously.

Further, the disclosed method may be associated with your color analysis process that may be classified into six parts that may include Color Wisdom History, Calendar, Your Color History, Your Color Body Map, Your Cartesian Analyses, and Clothing Summary. Further, for color wisdom history, the user may see their past data for notifications consisting of positive quotes/statements for maintaining their mental health and embracing their positive color into their daily life, that may be generated by the app algorithm. Based on the user profile, guided statement sets are generated which promotes positivity. The notifications also suggest the user with the adoption of their positive colors in their daily life. Further, in the display interface, the user may access a calendar that may be developed by the app algorithm based on their past data analyses. If any user wants to know their activity on a particular date then the user may access that data through the calendar by tapping on that particular date. It will show the tasks taken on that day, and the user can view the history of that day. With the help of this feature, the users may track their past activity. Further, using your color history, the user may see their past data analyses through a graphical plot in the display interface. Further, the app algorithm generates a graphical analysis where the number of occurrences is plotted in the X-axis and the color of clothing & accessory is plotted in the Y-axis. Further, all these data may be plotted with respect to the time metric. With the help of this analysis, users can see how many times they wear their positive color at a particular time. Further, the user may see monthly and yearly data analysis. Further, upon using Your Color Body Map, the users are asked to choose a body part shown in the display where they want to feel a particular color which they choose in the ongoing process. This is an analyzing method, with the help of users' response app algorithm can guide them for an easy and quick option of carrying their positive colors and thus closure with them, via finding apparel and accessories of their generated positive colors. Further, upon using Your Cartesian Analyses, the users may see a graphical interface where their positive color which is evaluated through the app algorithm shown as a background of the plot along with the statements they chose, and the intensity of its impact mapped on a single graph. The analysis of the previous selection that the user has done, is carried out through the algorithm (CAA).

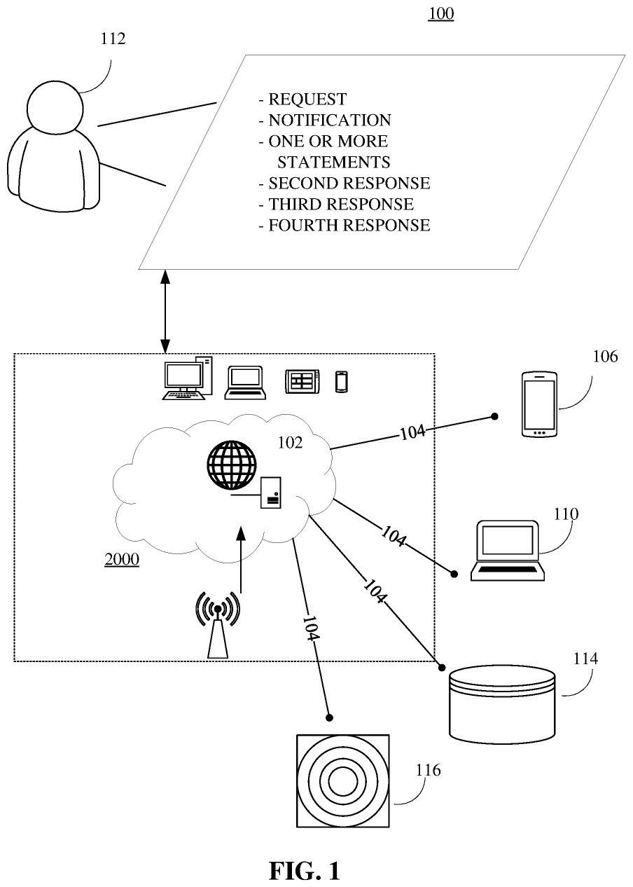

FIG. 1 is an illustration of an online platform 100 consistent with various embodiments of the present disclosure. By way of non-limiting example, the online platform 100 to facilitate self-analysis through data based on inputs associated with psychological states of a user may be hosted on a centralized server 102, such as, for example, a cloud computing service. The centralized server 102 may communicate with other network entities, such as, for example, a mobile device 106 (such as a smartphone, a laptop, a tablet computer, etc.), other electronic devices 110 (such as desktop computers, server computers, etc.), databases 114, and sensors 116 over a communication network 104, such as, but not limited to, the Internet. Further, users of the online platform 100 may include relevant parties such as, but not limited to, end-users, administrators, service providers, service consumers, and so on. Accordingly, in some instances, electronic devices operated by the one or more relevant parties may be in communication with the platform.

A user 112, such as the one or more relevant parties, may access online platform 100 through a web-based software application or browser. The web-based software application may be embodied as, for example, but not be limited to, a website, a web application, a desktop application, and a mobile application compatible with a computing device 2000.

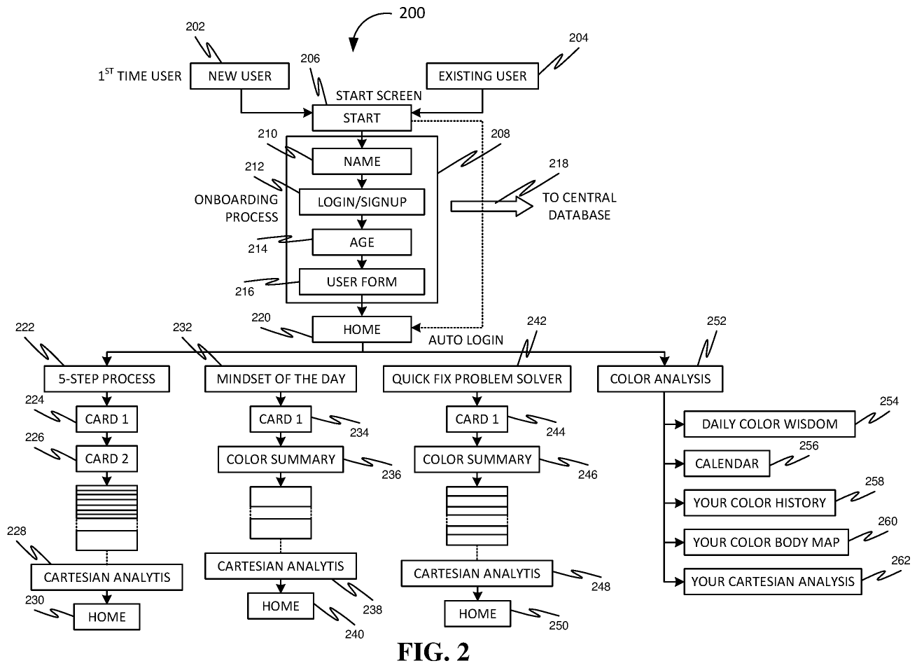

FIG. 2 is a flow diagram of a method 200 to facilitate self-analysis through data based on inputs associated with psychological states of a user, in accordance with some embodiments. Further, the method 200 recommends specific colored products based on a psychology of the user. Further, the data is derived from algorithms. Further, the method 200 may be performed by at least one of a new user 202 and an existing user 204. Further, at 206, the method 200 may include start. Further, the method 200 may be associated with an onboarding process 208. Further, at least one of the new user 202 and the existing user 204 may be associated with a name 210. Further, the method 200 may include login/sign up 212. Further, at least one of the new user 202 and the existing user 204 may be associated with an age 214. Further, at least one of the new user 202 and the existing user 204 may be associated with a user form 216. Further, at 218, the method 200 may include sending the onboarding process to a central database. Further, at 220, the method 200 may include auto logging in to home. Further, the method 200 may include a 5 step process 222. Further, at 224, the method 200 may be associated with a card 1. Further, at 226, the method 200 may be associated with a card 2. Further, at 228, the method 200 may include performing cartesian analysis. Further, at 230, the method 200 may include home. Further, after 220, at 232, the method 200 may be associated with a mindset of the day process. Further, at 234, the method 200 may be associated with a card 1. Further, at 236, the method 200 may include generating a card summary. Further, at 238, the method 200 may include performing cartesian analysis. Further, at 240, the method 200 may include home. Further, after 220, at 242, the method 200 may be associated with a quick fix problem solver process. Further, at 244, the method 200 may be associated with a card 1. Further, at 246, the method 200 may include generating a card summary. Further, at 248, the method 200 may include performing cartesian analysis. Further, at 250, the method 200 may include home. Further, after 220, at 252, the method 200 may include performing color analysis. Further, the color analysis may facilitate allowing a user to view a daily color wisdom 254, a calendar 256, your color history 258, your color body map 260, and your cartesian analysis 262.

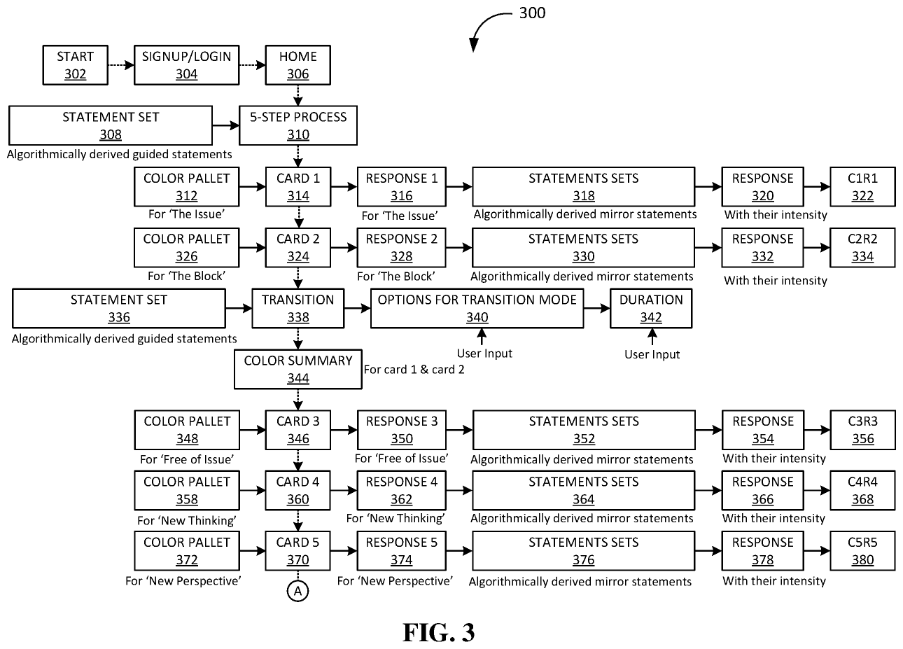

FIG. 3 is a flow diagram of a method 300 to facilitate self-analysis through data based on inputs associated with psychological states of a user, in accordance with some embodiments. Accordingly, the method 300 may be associated with a colornoustics process (5-step process). Further, 302, the method 300 may include start. Further, at 304, the method 300 may include signup/login. Further, 306, the method 300 may include a home. Further, at 308, the method 300 may be associated with statement sets. Further, the statement sets may include algorithmically derived guided statements. Further, at 310, the method 300 may include a 5-step process. Further, the method 300 may be associated with a color pallet 312 for “the issue”. Further, at 314, the method 300 may be associated with a card 1.

Further, for a first color associated with the card 1, the users may be asked to think about the problem that is going on in their mind without trying to solve it or explain it, while looking at the 20 colors displayed on the screen, intuitively select a color which they dislike the most at that particular moment. Further, at 316, the users may exert a response 1 intuitively by choosing a color that they dislike the most at that particular moment. Further, at 318, the users may be asked to choose one or more statements that reflect their current state of mind (what they often think to themselves), along with the intensity of the statement on a scale of mild (ss=1), medium (ss=2), and strong (ss=3). Further, at 320, a response may be fetched by the app algorithm and stored in the memory database for future usage. Further, at 322, the method 300 may be associated with C1R1.

Further, at 324, the method 300 may be associated with a card 2. Further, at 326, the method 300 may be associated with a color pallet for “the block”. Further, for card 2, the users (or users) may be asked to think about the probable reasons for their issues that are blocking them psychologically while looking at 20 colors displayed on a screen. Further, the users may have to pick their least favorite color at that particular moment. Further, the color that the users chose previously in Card 1 is not available in the selection options. Further, for a response for the card 2, at 328, the users may exert a response 2 intuitively by choosing a color which the user may dislike the most at that particular moment for the Card. Further, at 330, the user may be asked to choose one or more statements that represent their current state of mind, along with the intensity of the statement on a scale of mild (ss=1), medium (ss=2), and strong (ss=3). Further, at 332, a response for the card 2 may be fetched by the app algorithm and stored in a memory database for future usage. Further, at 334, the method 300 may be associated with a C2R2. Further, the colornostics process may include preparation. Further, in the display interface, algorithmically derived guided messages are shown which guides the user to optimize the treatment procedure. Further, at 336, the method 300 may be associated with statement sets. In this part, the user may sit comfortably, take a breath, and feel relaxed in front of their device screens. Further, at 338, the method 300 may include a transition state. Further, at 340, the method 300 may be associated with options for transition mode. Further, in the transition state, algorithmically derived messages guide the users, where users have to release all those mental ailments which block them to maintain a healthy mental state and are prepared to be mentally free themselves from those issues. In this transition state (or period), an audiovisual clip plays on the device screen, where the color transition takes place with respective audio backgrounds that may enhance the treatment procedure. Further, the colornostics process may include a response for transition. Further, five options may be given for the user to spontaneously select the transition modes from waterfall, beach, mountain top, forest, and fire. Further, at 342, the user may select a duration of the transition. Further, colors that may be selected in the previous stages as the card 1 and the card 2 are seen here vanishing with the transition mode selected by the user in the 30 seconds/60-second video. Further, at 344, the colornostics process may be associated with a color summary. Further, in the display interface, the color summary may be displayed. Further, the users may see the color summary of the colors that the user may choose for the card 1 and the card 2. Further, at 346, the method 300 may be associated with a card 3. After completing the transition state (or period), the users may be asked to think about their state of mind if their issue is resolved and the user is free of their issues, while looking at 20 (here the color that they chose previously in the Cards 1, 2 is not available in the selection options) colors associated with a color pallet 348 displayed on the screen, instinctually select a color for the card 3, which they like the most at that particular moment. Further, for a response for the card 3, at 350, the users may exert a response 3 intuitively by choosing a color which they like the most for the card 3 at that particular moment. While selecting the color, at 352, the user may select the one or more statements that represent their current state of mind along with the intensity of the statement on a scale of mild (ss=1), medium (ss=2), and strong (ss=3). Further, at 354, a response may be fetched by the app algorithm and stored in the memory database for future usage. Further, at 356, the method 300 may be associated with a C3R3. Further, for a card 4, the users may be asked to think about their newly developed mindset that has been activated in their new perspective, while looking at 20 colors (here the color that they chose previously in Cards 1, 2, 3 is not available in the selection options) from a color pallet 358 displayed on the screen, intuitively select a color (the Card 4) which they like the most at that particular moment. Further, at 360, the method 300 may be associated with the card 4. Further, for a response for the card 4, at 362, the users may exert a response 5 intuitively by choosing a color which they like the most for the card 4 at that particular moment and the response 5 is fetched by the app algorithm and stored in the memory database for future usage. While selecting the most favorite color, at 364, the user may have to select one or more statements that represent their current state of mind along with the intensity of the statement on a scale of mild (ss=1), medium (ss=2), and strong (ss=3). Further, at 366, a response is fetched by the app algorithm and stored in the memory database for future usage. Further, at 368, the method 300 may be associated with C4R4. Further, at 370, the method 300 may be associated with a card 5. Further, for the card 5, the users may be asked to think about their ongoing growth and mental sanity, while looking at 20 colors (here the color that they chose previously in Cards 1, 2, 3, 4 is not available in the selection options) from a color pallet 372 displayed on the screen, intuitively select a color (for the card 5) which they like the most at that particular moment. Further, for a response for the card 5, at 374, the users may exert a response 5 intuitively by choosing a color which they like the most for the card 5 at that time. While selecting the color, at 376, the users may select one or more statements that represent their current state of mind along with the intensity of the statement on a scale of mild (ss=1), medium (ss=2), and strong (ss=3). Further, at 378, a response may be fetched by the app algorithm and stored in the memory database for future usage. Further, at 380, the method 300 may be associated with C4R4. Further, the colornostics process may be associated with a color summary 382 for 3, 4, 5. Further, in the display interface, the color summary 382 may be displayed. Further, the users may see the color summary 382 of the colors that they chose for the card 3, the card 4, and the card 5. Further, at 384, the users may see the colors that the user chose for the card 3 using the C3R3. Further, at 386, the user may view a color summary for the card 3. Further, at 388, the method 300 may include receiving user input for color body map 390 (free of issue). Further, the user may be asked to choose a body part shown in the display interface where they feel that color which they choose for completing the procedure of color body mapping. Further, for a response for Color Body Map, the users may exert their response intuitively by choosing the body parts and the response is shown through the Color Body Map. Further, at 392, the users may see the colors that they chose for the card 4 using the C4R4. Further, at 394, the user may view the color summary for the card 4 (new thinking). Further, at 396, the method 300 may include receiving a user input for a color body map for new thinking 398. Further, the user may be asked to choose a body part shown in the display interface where the user may feel that color for completing the color body mapping. Further, for a response for the Color Body Map, the users may exert their response intuitively by choosing the body parts and the response is shown through the Color Body Map. Further, at 3100, the users may see the colors that they chose for the card 5 using the C5R5. Further, at 3102, the user may view a color summary for the card 5 (new perspective). Further, at 3104, the method 300 may include receiving user input for a color body map for new perspective 3106. Further, the user may be asked to choose a body part shown in the display interface where they feel that color for completing the color body mapping. Further, for a response for the Color body map: The users exert their response intuitively by choosing the body parts and the response is shown through the Color Body Map. After going through this whole procedure where users are asked to select a body location where they feel the colors in Card 3, Card 4, and Card 5. The location on the body may be a head area, chest area, stomach area, thighs area, legs area, hands area. The users may exert their response and the summary for the same is displayed simultaneously. The response is fetched by the app algorithm and stored in the memory database for future usage. Further, at 3108, the method 300 may be associated with statement sets (or algorithmically derived statements). Further, at 3110, the method 300 may be associated with a summary. Further, the algorithmically derived statements may be shown in this section which indicates the completion of the procedure. Further, at 3112, the method 300 may include performing cartesian analysis. Further, at 3114, the method 300 may be associated with home. Further, the cartesian analysis may be performed for free of issue 3116. Further, the cartesian analysis may be performed for new thinking 3118. Further, the cartesian analysis may be performed for new perspective 3120. Further, the analysis of the previous selection that the user has done may be carried out through the App Algorithm (CAA). Further, the user may see their choice of color along with the statements they chose and the intensity of its impact mapped on a single graph. This is shown for the cards 3, 4 and 5, this is stored with a value of time, which can be further mapped and presented to the user.

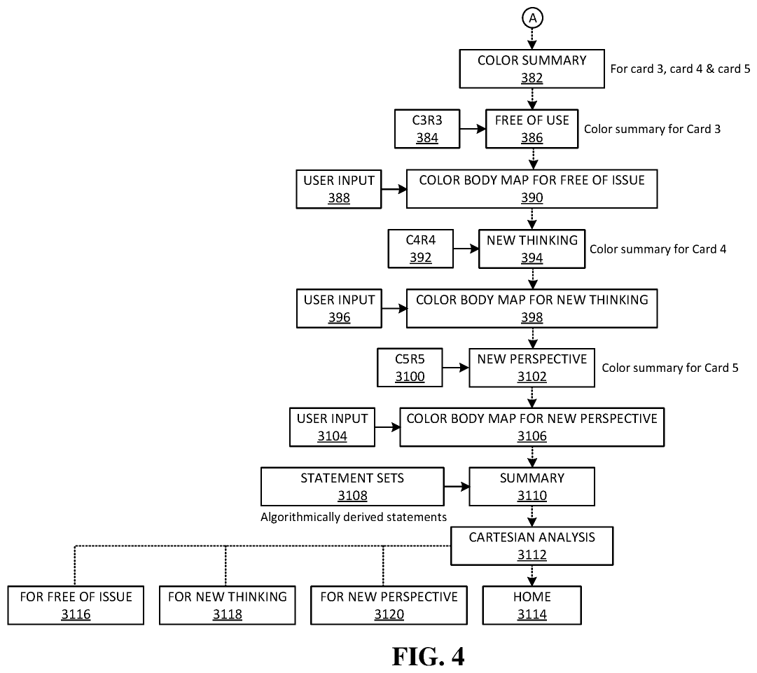

FIG. 4 is a continuation flow diagram of FIG. 3.

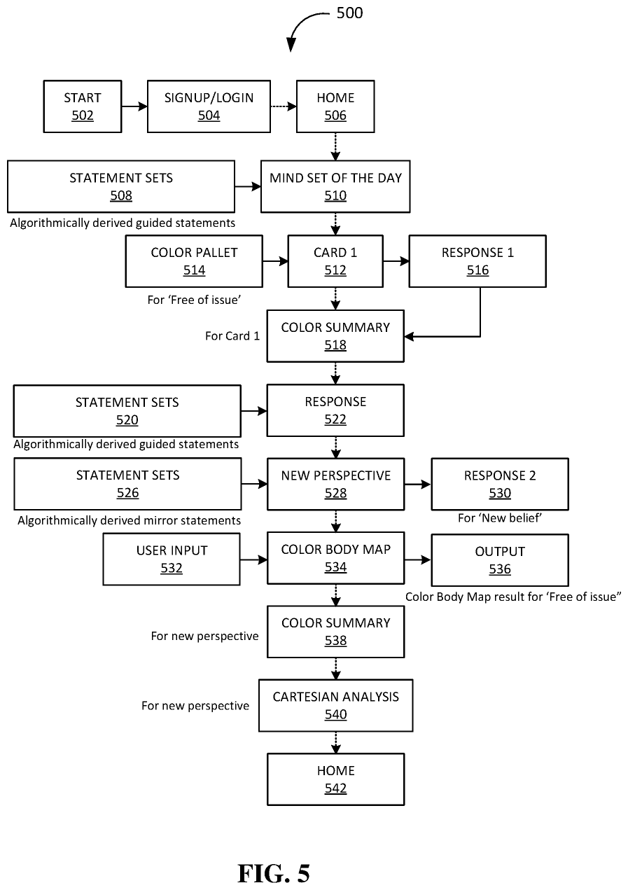

FIG. 5 is a flow diagram of a method 500 to facilitate self-analysis through data based on inputs associated with psychological states of a user, in accordance with some embodiments. Accordingly, the method 500 may be associated with a mindset of the day process (1-step process). Further, 502, the method 500 may include start. Further, 504, the method 500 may include signup/login. Further, 506, the method 500 may include a home. Further, at 508, the method 500 may be associated with statement sets. Further, the statement sets may include algorithmically derived guided statements. Further, at 510, the method 500 may be associated with the mindset of the day process.

Further, at 512, the method 500 may be associated with a card 1. Further, for a color for the card 1, the users may be asked to think about a mindset that they want for the day, while looking at the 20 colors from a color pallet 514 displayed on the screen, intuitively select a color. Further, for a response for the card 1, at 516, the users may exert a response intuitively by choosing a color that represents their mindset for the day. Further, for a color Summary for Card 1, at 518, the selected color may be displayed here in the section of the color summary. Further, algorithmically derived messages may be shown in this section. Further, at 520, the users may be asked to select one or more statements that represent their current state of mind along with the intensity of the statement on a scale of mild (ss=1), medium (ss=2), and strong (ss=3). Further, at 522, the users may exert a response by selecting these statements which described their new thought (perspective), and the response is fetched by the app algorithm and stored in the memory database for future usage. Further, at 526, the method 500 may be associated with statement sets. Further, at 528, the method 500 may be associated with a new perspective of the user. Further, at 530, the method 500 may include receiving a response 2 of the user. Further, at 532, the method 500 may include receiving user input for a color body map.

Further, the users may be asked to choose a body part shown in the display where they want to feel the previous color. Further, for a response for the color Body Map, the users may exert their response intuitively by choosing a body part that described their current state of mind. Further, at 534, the response is shown through the Color Body Map. Further, at 536, the method 500 may include an output associated with the color body map result for “free of issue”. In the display interface, at 538, the color summary may be displayed. Further, at 540, the method 500 may include performing cartesian analysis. Further, at 542, the method 500 may include home. Further, the users may see the colors that they chose for Card 1 which represents their new thought. The analysis of the previous selection that the user has done may be carried out through Colornostics App Algorithm (CAA). Further, the user may see their choice of color along with the statements they chose and the intensity of its impact mapped on a single graph. This is shown for Card 1.

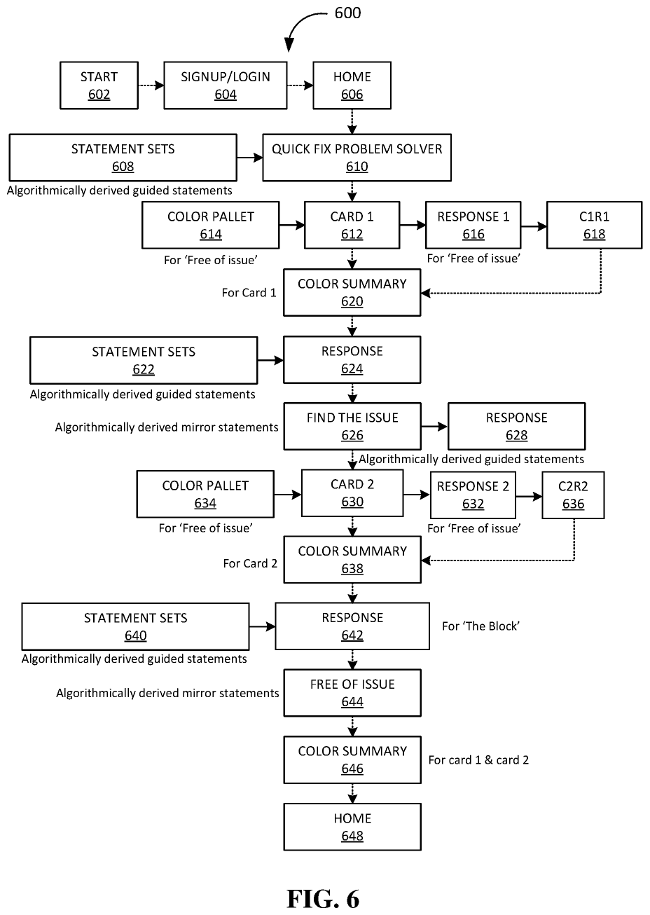

FIG. 6 is a flow diagram of a method 600 to facilitate self-analysis through data based on inputs associated with psychological states of a user, in accordance with some embodiments. Accordingly, the method 600 may be associated with a quick-fix problem solver process that may be a two-step process. Accordingly, 602, the method 600 may include start. Further, 604, the method 600 may include signup/login. Further, 606, the method 600 may include a home. Further, at 608, the method 600 may be associated with statement sets. Further, the statement sets may include algorithmically derived guided statements. Further, at 610, the method 600 may be associated with the quick fix problem solver process.

Further, at 612, the method 600 may be associated with a card 1. Further, for the first color (card 1), the users may be asked to think about the trouble that is ongoing in their mind without trying to solve it or explain it, while looking at the 20 colors from a color pallet 614 displayed on the screen, intuitively select a color which they dislike the most at that particular moment. Further, for a response for the card 1, at 616, the users may exert a response 1 intuitively by choosing a color which they dislike the most at that particular moment. Further, at 618, the method 600 may be associated with C1R1. Further, at 620, the selected color (or color) may be displayed here as a color summary. Further, at 622, algorithmically derived messages may be shown in this section and the users may be asked to select one or more statements from the given list which is relatable most deeply with their ongoing trouble along with the intensity of the statement on a scale of mild (ss=1), medium (ss=2), and strong (ss=3). Further, at 624, the users exert a response by selecting the statements that represent their current state of mind, and the response is fetched by the app algorithm and stored in the memory database for future usage. Further, at 626, the method 600 may include finding an issue. Further, at 628, the method 600 may include receiving a response. Further, at 630, the method 600 may be associated with a card 2. Further, for the card 2, the users may be asked to think about their state of mind if their issue is resolved and the users may be free of their issues while looking at the colors in the display. Further, the users may be asked to choose a color that represents their new experience. Further, at 632, for a response for Card 2, the users may exert their response intuitively by choosing a color from a color pallet 634, which they think is best suitable for representing their new experience at that particular moment. Further, at 636, the method 600 may be associated with C2R2. Further, at 638, the color (or selected color) may be displayed here as a color summary. Further, at 640, algorithmically derived messages may be shown in this section. Further, at 642, the users are asked to select one statement from the given list which is relatable with their current state along with the intensity of the statement on a scale of mild (ss=1), medium (ss=2), and strong (ss=3). The users exert their response by selecting the statements that represent their current state of mind, and the response is fetched by the app algorithm and stored in the memory database for future usage. Further, at 644, the method 600 may include the user being free of an issue. This statement shows how they feel if the issue was solved. Further, in the display interface, at 646, the color summary may be displayed. Further, the users may see the colors that they chose for Card 1 & Card 2 they select previously. Further, at 648, the method 600 may be associated with home.

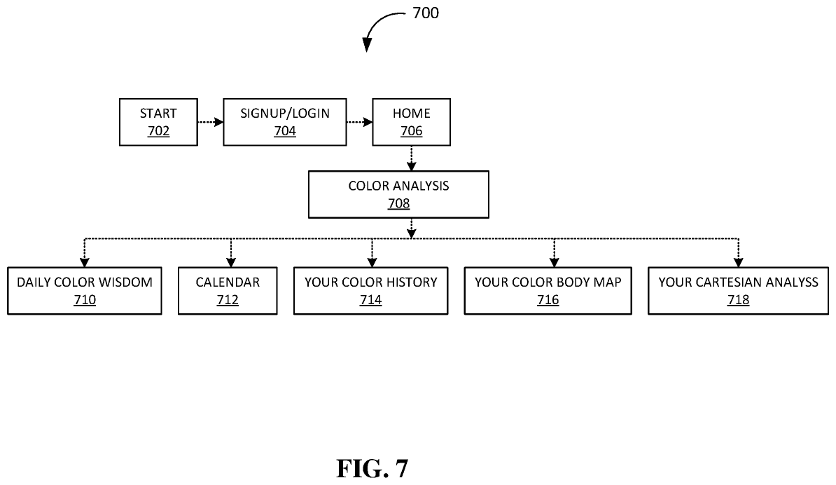

FIG. 7 is a flow diagram of a method 700 to facilitate self-analysis through data based on inputs associated with psychological states of a user, in accordance with some embodiments. Accordingly, the method 700 may include performing color analysis. Accordingly, 702, the method 700 may include start. Further, 704, the method 700 may include signup/login. Further, 706, the method 700 may include a home. Further, at 708, the method 700 may be associated with statement sets. Further, the color analysis may be associated with color wisdom 710, calendar 712, your color history 714, your color body map 716, and your cartesian analysis 718.

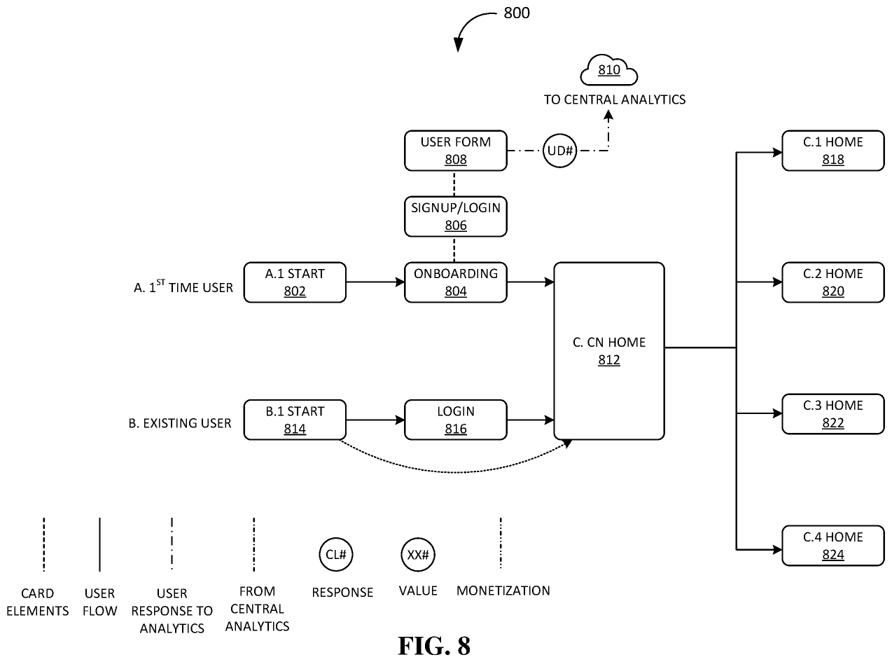

FIG. 8 is a flow diagram of a method 800 to facilitate self-analysis through data based on inputs associated with psychological states of a user, in accordance with some embodiments. Accordingly, at 802, the method 800 may include start by a first-time user. Further, at 804, the method 800 may include onboarding. Further, at 806, the method 800 may include signup/login. Further, at 808, the method 800 may include user data collection based on a user form. Further, at 810, the method 800 may include sending a user response to central analytics. Further, after 804, at 812, the method 800 may include Colornostics (CN) home.

Further, at 814, the method 800 may include start by an existing user. Further, at 816, the method 800 may include login by the existing user. Further, after 816, the method 800 may proceed to 812. Further, after 814, the method 800 may proceed to 812 based on auto-login. Further, the CN home may be associated with the 5-step process, mindset of the data process, quick-fix problem solver process, and the color analysis process.

Further, at 818, the method 800 may include a home associated with the 5-step process for providing instructions for use. Further, at 820, the method 800 may include a home associated with the mindset of the day process for providing instructions for use. Further, at 822, the method 800 may include a home associated with the quick fix problem solver process for providing instructions for use. Further, at 824, the method 800 may include a home associated with the color analysis process for providing instructions for use.

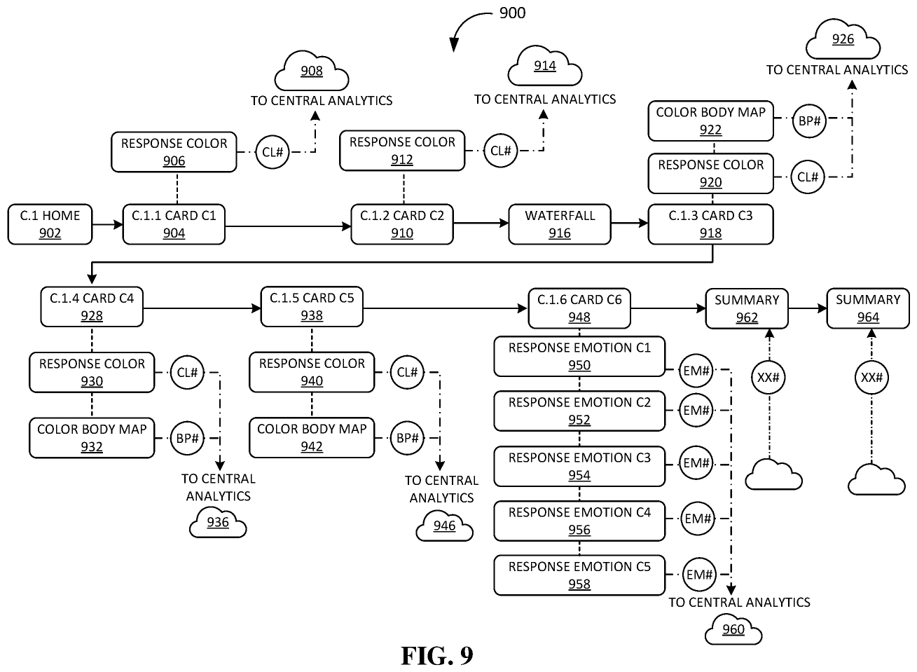

FIG. 9 is a flow diagram of a method 900 to facilitate self-analysis through data based on inputs associated with psychological states of a user, in accordance with some embodiments. Accordingly, at 902, the method 900 may include a home associated with the 5-step process. Further, at 904, the method 900 may include issuing a card C1 and instruction statements. Further, the card C1 may be associated with a response color 906. Further, at 908, the method 900 may include sending a user response to central analytics. Further, at 910, the method 900 may be associated with a blocking card C2 and instruction statements. Further, the card C2 may be associated with a response color 912. Further, at 914, the method 900 may include sending a user response to central analytics. Further, at 916, the method 900 may be associated with the waterfall with audio. Further, at 918, the method 900 may be associated with a card 3. Further, the user may receive instruction statements associated with the card 3 (free of issue). Further, the card C3 may be associated with a response color 920. Further, at 922, the response color may be associated with a color body map. Further, the color body map may be associated with a location on a body. Further, at 926, the color body map and the response color may be transmitted to central analytics. Further, after 918, at 928, the method 900 may be associated with a card 4. Further, at 930, the card 4 may be associated with a response color. Further, at 932, the response color associated with the card 4 may be associated with a color body map. Further, the color body map associated with the card 4 may be associated with a location on a body. Further, at 936, the method 900 may include the color body map and the response color associated with the card 4 may be transmitted to the central analytics.

Further, after 928, at 938, the method 900 may be associated with a card 5. Further, at 940, the card 5 may be associated with a response color. Further, at 942, the response color associated with the card 5 may be associated with a color body map. Further, the color body map associated with the card 5 may be associated with a location on a body. Further, at 946, the method 900 may include transmitting the color body map and the response color associated with the card 5 to the central analytics.

Further, after 938, at 948, the method 900 may be associated with a card 6. Further, the card 6 may be associated with a summary. Further, at 950, the card 6 may be associated with a response emotion C1. Further, at 952, the card 6 may be associated with a response emotion C2. Further, at 954, the card 6 may be associated with a response emotion C3. Further, at 956, the card 6 may be associated with a response emotion C4. Further, at 958, the card 6 may be associated with a response emotion C5. Further, at 960, the method 900 may include transmitting the response emotion 1, the response emotion 2, the response emotion 3, the response emotion 4, and the response emotion 5 associated with the card 6 to the central analytics. Further, at 962, the method 900 may include receiving a summary associated with user choices from the central analytics. Further, at 964, the method 900 may include a summary associated with cartesian maps from the central analytics.

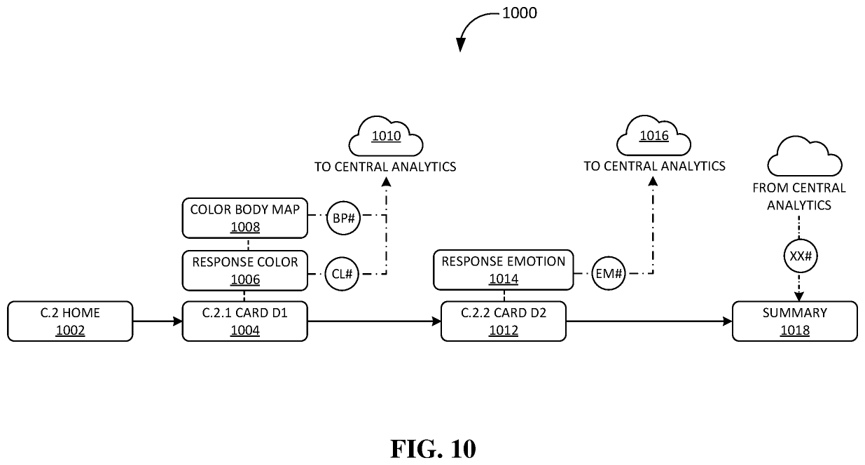

FIG. 10 is a flow diagram of a method 1000 to facilitate self-analysis through data based on inputs associated with psychological states of a user, in accordance with some embodiments. Accordingly, the method 1000 may be associated with the mindset of the day process. Further, at 1002, the method 1000 may include a home associated with the mindset of the day process. Further, the home may provide instruction for use. Further, at 1004, the method 1000 may include a dialog card D1. Further, the card D1 may be associated with a response color 1006. Further, the card D1 may be associated with a color body map 1008 that may be associated with a location on a body. Further, at 1010, the method 1000 may include sending the response color and the color body map to central analytics. Further, at 1012, the method 1000 may include a card D2. Further, at 1014, the card D2 may be associated with a response emotion. Further, at 1016, the method 1000 may include sending the response emotion and the color body map to central analytics. Further, at 1018, the method 1000 may include receiving a summary associated with an end result from the central analytics.

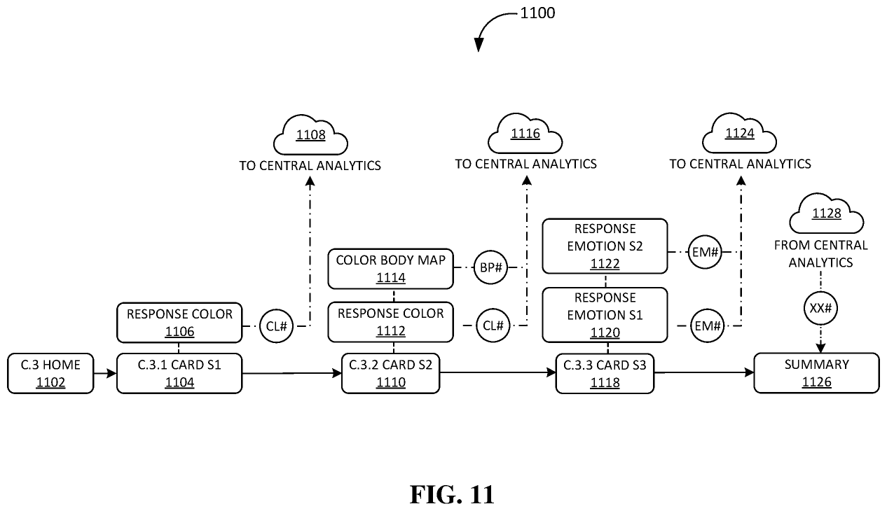

FIG. 11 is a flow diagram of a method 1100 to facilitate self-analysis through data based on inputs associated with psychological states of a user, in accordance with some embodiments. Accordingly, the method 1100 may be associated with the quick fix problem solver process. Further, at 1102, the method 1100 may include a home associated with the quick fix problem solver process. Further, the home may provide instruction for use. Further, at 1104, the method 1100 may include a special even card S1. Further, the card Si may be associated with a response color 1106. Further, the card S1 may facilitate understanding the problem. Further, at 1108, the method 1100 may include sending the response color to central analytics. Further, at 1110, the method 1100 may include a card S2. Further, at 1112, the card S2 may be associated with a response color. Further, at 1114, the card S2 may be associated with a color body map. Further, at 1116, the method 1100 may include sending the response color and the color body map to central analytics. Further, the card S2 may facilitate unlocking new thoughts, feelings, perspectives, and solutions. Further, at 1118, the method 1100 may include a card S3. Further, at 1120, the card S2 may be associated with a response emotion—S1. Further, at 1122, the card S3 may be associated with a response emotion—S2. Further, at 1124, the method 1100 may include sending the response emotion S1 and the response emotion S2 to the central analytics. Further, at 1126, the method 1100 may include a summary from central analytics 1128.

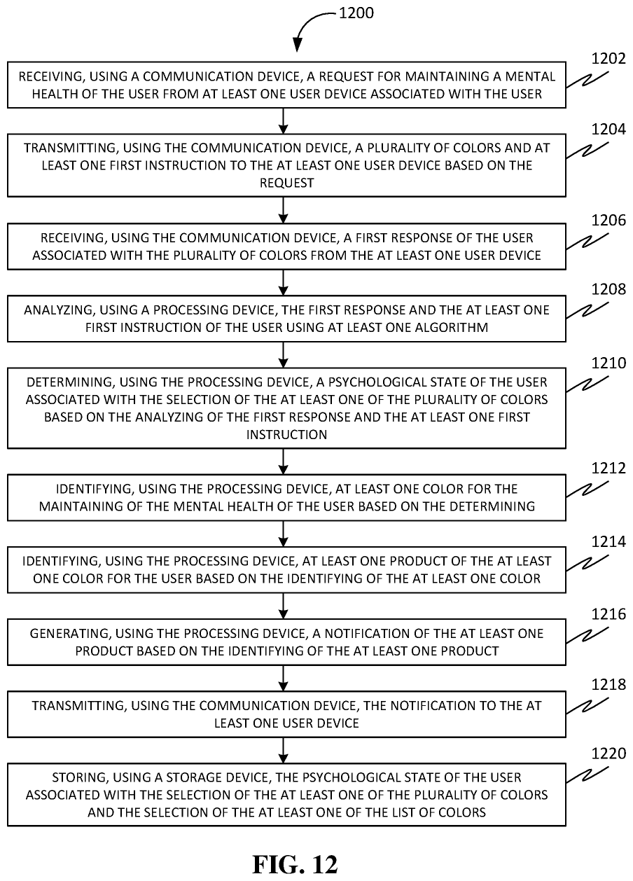

FIG. 12 is a flow chart of a method 1200 to facilitate self-analysis through data based on inputs associated with psychological states of a user, in accordance with some embodiments. Further, the data is derived from algorithms. Further, the method 1200 recommends specific colored products based on a psychology of the user. Further, at 1202, the method 1200 may include receiving, using a communication device (such as a communication device 1802), a request for maintaining a mental health of the user from at least one user device (such as at least one user device 1902) associated with the user. Further, at 1204, the method 1200 may include transmitting, using the communication device, a plurality of colors and at least one first instruction to the at least one user device based on the request. Further, the at least one user device may include at least one presentation device. Further, the at least one presentation device may be configured for presenting the at least one instruction and the plurality of colors to the user. Further, the at least one presentation device may be a display device, a speaker, a projector, etc. Further, at 1206, the method 1200 may include receiving, using the communication device, a first response of the user associated with the plurality of colors from the at least one user device. Further, the first response may include a selection of at least one of the plurality of colors and a first reaction of the user associated with the selection of the at least one of the plurality of colors. Further, the first reaction may include an unconscious reaction. Further, the first reaction may include an unconscious physical response, an unconscious physiological response, an emotional response, etc. of the user based on the plurality of colors. Further, the unconscious physical response may include unconscious hand gestures, unconscious facial expressions, unconscious eye movements, unconscious eyelids movements, etc. Further, the physiological response may include a change in a heartbeat, an unconscious change in a respiration rate, etc. Further, at 1208, the method 1200 may include analyzing, using a processing device (such as a processing device 1804), the first response and the at least one first instruction of the user using at least one algorithm. Further, at 1210, the method 1200 may include determining, using the processing device, a psychological state of the user associated with the selection of the at least one of the plurality of colors based on the analyzing of the first response and the at least one first instruction. Further, the psychological state may include a mental state of the user, an emotional state of the user, etc. Further, the psychological statepsychological state may include a positive psychological state and a negative psychological state. Further, the psychological state may include a happy state, a sad state, an anxious state, an anger sate, a depressed state, etc. Further, at 1212, the method 1200 may include identifying, using the processing device, at least one color for the maintaining of the mental health of the user based on the determining. Further, at 1214, the method 1200 may include identifying, using the processing device, at least one product of the at least one color for the user based on the identifying of the at least one color. Further, the at least one product may include an item, an article, an object, an apparel, etc. Further, at 1216, the method 1200 may include generating, using the processing device, a notification of the at least one product based on the identifying of the at least one product. Further, at 1218, the method 1200 may include transmitting, using the communication device, the notification to the at least one user device. Further, at 1220, the method 1200 may include storing, using a storage device (such as a storage device 1806), the psychological state of the user associated with the selection of the at least one of the plurality of colors and the selection of the at least one of the list of colors.

Further, in some embodiments, the at least one user device may include at least one sensor. Further, the at least one sensor may include an image sensor, a microphone, a biological sensor, etc. Further, the at least one sensor may be configured for generating the first reaction of the user based on detecting at least one of a physiological state, a physical state, and an emotional state of the user during viewing the plurality of colors.

Further, in some embodiments, the at least one user device may include at least one input device. Further, the at least one input device may be configured for generating the selection of the at least one of the plurality of colors based on receiving at least one user input action. Further, the at least one user input action may include a conscious reaction of the user. Further, the conscious reaction may include a conscious hand gesture, a conscious facial expression, a conscious respiration rate, etc.

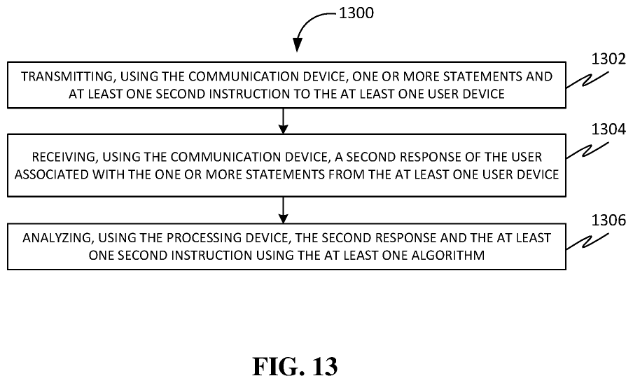

FIG. 13 is a flow chart of a method 1300 to facilitate self-analysis through data based on inputs associated with psychological states of a user, in accordance with some embodiments. Accordingly, at 1302, the method 1300 may include transmitting, using the communication device, one or more statements and at least one second instruction to the at least one user device. Further, the at least one presentation device may be configured for presenting the one or more statements and the at least one second information to the user. Further, at 1304, the method 1300 may include receiving, using the communication device, a second response of the user associated with the one or more statements from the at least one user device. Further, the second response may include a selection of at least one of the one or more statements and a second reaction of the user associated with the selection of the at least one of the one or more statements. Further, the second reaction may include an unconscious reaction. Further, the second reaction may include an unconscious physical response, an unconscious physiological response, an emotional response, etc. of the user based on the plurality of colors. Further, the unconscious physical response may include unconscious hand gestures, unconscious facial expressions, unconscious eye movements, unconscious eyelids movements, etc. Further, the physiological response may include a change in a heartbeat, an unconscious change in a respiration rate, etc. Further, at 1306, the method 1300 may include analyzing, using the processing device, the second response and the at least one second instruction using the at least one algorithm. Further, the determining of the psychological state of the user may be based on the analyzing of the second response and the at least one second instruction.

Further, in some embodiments, the method 1300 may include generating, using the processing device, the one or more statements using at least one first algorithm based on the analyzing of the first response and the at least one first instruction. Further, the one or more statements corresponds to the selection of the at least one of the plurality of colors.

Further, in some embodiments, the at least one user device may include at least one first sensor. Further, the at least one first sensor may include an image sensor, a microphone, a biological sensor, etc. Further, the at least one first sensor may be configured for generating the second reaction of the user based on detecting at least one of a physiological state, a physical state, and an emotional state of the user during viewing the one or more statements.

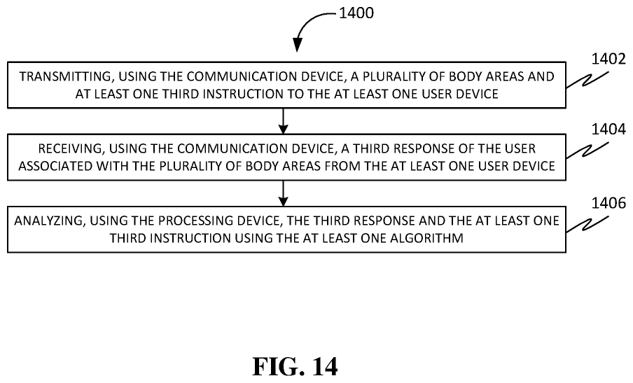

FIG. 14 is a flow chart of a method 1400 to facilitate self-analysis through data based on inputs associated with psychological states of a user, in accordance with some embodiments. Further, at 1402, the method 1400 may include transmitting, using the communication device, a plurality of body areas and at least one third instruction to the at least one user device. Further, the plurality of body areas may include a chest area, a back area, a waist area, a leg area, a foot area, a thigh area, a palm area, an arm area, a neck area, a belly area, a facial area, etc. Further, the at least one presentation device may be configured for presenting the plurality of body areas and the at least one third instruction. Further, at 1404, the method 1400 may include receiving, using the communication device, a third response of the user associated with the plurality of body areas from the at least one user device. Further, the third response may include a selection of at least one of the plurality of body areas and a third reaction associated with the selection of the at least one of the plurality of body areas. Further, at 1406, the method 1400 may include analyzing, using the processing device, the third response and the at least one third instruction using the at least one algorithm. Further, the identifying of the at least one product may be based on the analyzing of the third response and the at least one third instruction.

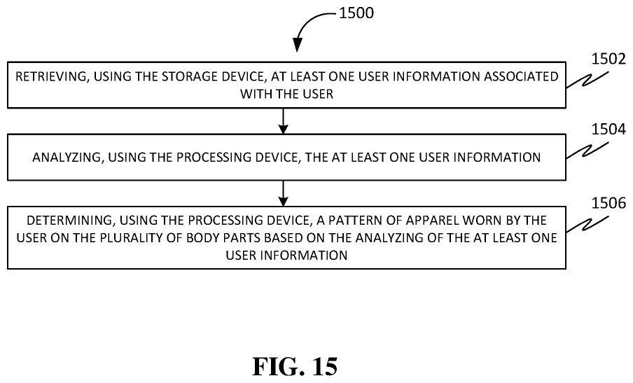

FIG. 15 is a flow chart of a method 1500 to facilitate self-analysis through data based on inputs associated with psychological states of a user, in accordance with some embodiments. Accordingly, at 1502, the method 1500 may include retrieving, using the storage device, at least one user information associated with the user. Further, the at least one user information may include a user's profile, a user's apparel selection, etc. Further, at 1504, the method 1500 may include analyzing, using the processing device, the at least one user information. Further, at 1506, the method 1500 may include determining, using the processing device, a pattern of apparel worn by the user on the plurality of body parts based on the analyzing of the at least one user information. Further, the at least one product may include at least one apparel. Further, the identifying of the at least one product may include identifying the at least one apparel of the at least one color for the user based on the determining of the pattern of apparel worn by the user.

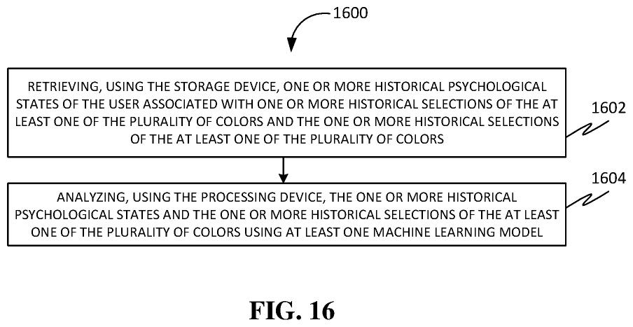

FIG. 16 is a flow chart of a method 1600 to facilitate self-analysis through data based on inputs associated with psychological states of a user, in accordance with some embodiments. Accordingly, at 1602, the method 1600 may include retrieving, using the storage device, one or more historical psychological states of the user associated with one or more historical selections of the at least one of the plurality of colors and the one or more historical selections of the at least one of the plurality of colors. Further, at 1604, the method 1600 may include analyzing, using the processing device, the one or more historical psychological states and the one or more historical selections of the at least one of the plurality of colors using at least one machine learning model. Further, the at least one machine learning model may be trained in pattern detection. Further, the determining of the psychological state of the user may be based on the analyzing of the one or more historical psychological states and the one or more historical selections of the at least one of the plurality of colors.

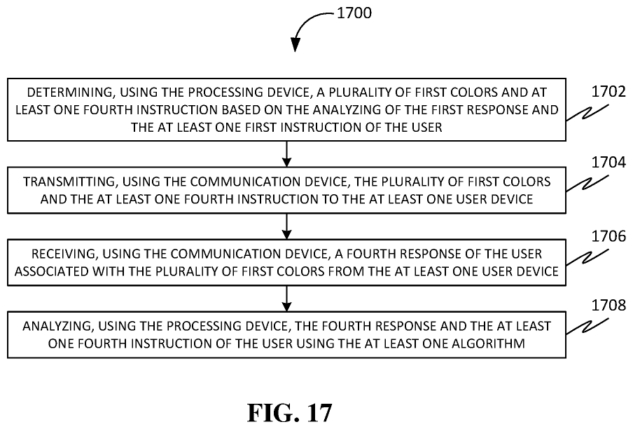

FIG. 17 is a flow chart of a method 1700 to facilitate self-analysis through data based on inputs associated with psychological states of a user, in accordance with some embodiments. Accordingly, at 1702, the method 1700 may include determining, using the processing device, a plurality of first colors and at least one fourth instruction based on the analyzing of the first response and the at least one first instruction of the user. Further, the plurality of first colors does not comprise the selection of the at least one of the plurality of colors. Further, at 1704, the method 1700 may include transmitting, using the communication device, the plurality of first colors and the at least one fourth instruction to the at least one user device. Further, the at least one presentation device may be configured for presenting the plurality of first colors and the at least one fourth instruction to the user. Further, at 1706, the method 1700 may include receiving, using the communication device, a fourth response of the user associated with the plurality of first colors from the at least one user device. Further, the fourth response may include a selection of at least one of the plurality of first colors and a fourth reaction of the user associated with the selection of the at least one of the plurality of first colors. Further, at 1708, the method 1700 may include analyzing, using the processing device, the fourth response and the at least one fourth instruction of the user using the at least one algorithm. Further, the determining of the psychological state of the user may be based on the analyzing of the fourth response and the at least one fourth instruction.

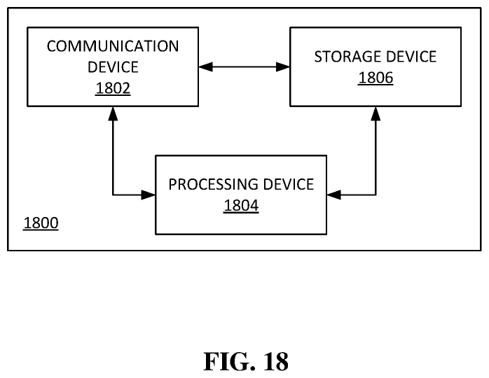

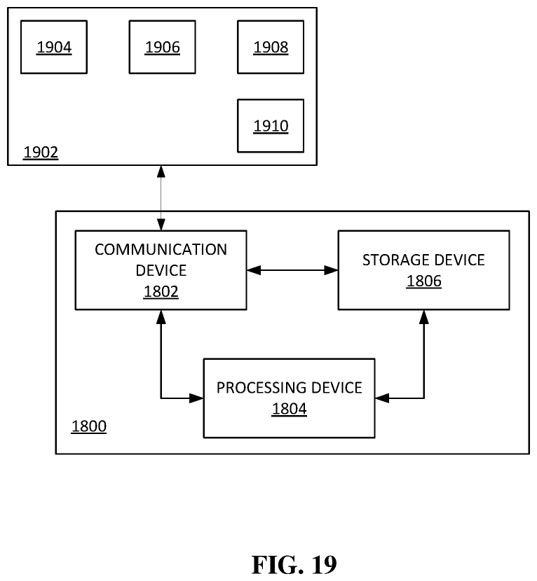

FIG. 18 is a block diagram of a system 1800 to facilitate self-analysis through data based on inputs associated with psychological states of a user, in accordance with some embodiments. Further, the data is derived from algorithms. Further, the system 1800 recommends specific colored products based on a psychology of the user. Further, the system 1800 may include a communication device 1802 configured for receiving a request for maintaining a mental health of the user from at least one user device 1902 (as shown in FIG. 19) associated with the user. Further, the communication device 1802 may be configured for transmitting a plurality of colors and at least one first instruction to the at least one user device 1902 based on the request. Further, the at least one user device 1902 may include at least one presentation device 1904 (as shown in FIG. 19). Further, the at least one presentation device 1904 may be configured for presenting the at least one instruction and the plurality of colors to the user. Further, the communication device 1802 may be configured for receiving a first response of the user associated with the plurality of colors from the at least one user device 1902. Further, the first response may include a selection of at least one of the plurality of colors and a first reaction of the user associated with the selection of the at least one of the plurality of colors. Further, the communication device 1802 may be configured for transmitting a notification to the at least one user device 1902. Further, the system 1800 may include a processing device 1804 communicatively coupled with the communication device 1802. Further, the processing device 1804 may be configured for analyzing the first response and the at least one first instruction of the user using at least one algorithm. Further, the processing device 1804 may be configured for determining a psychological state of the user associated with the selection of the at least one of the plurality of colors based on the analyzing of the first response and the at least one first instruction. Further, the processing device 1804 may be configured for identifying at least one color for the maintaining of the mental health of the user based on the determining. Further, the processing device 1804 may be configured for identifying at least one product of the at least one color for the user based on the identifying of the at least one color. Further, the processing device 1804 may be configured for generating the notification of the at least one product based on the identifying of the at least one product. Further, the system 1800 may include a storage device 1806 communicatively coupled with the processing device 1804. Further, the storage device 1806 may be configured for storing the psychological state of the user associated with the selection of the at least one of the plurality of colors and the selection of the at least one of the list of colors.

Further, in some embodiments, the communication device 1802 may be configured for transmitting one or more statements and at least one second instruction to the at least one user device 1902. Further, the at least one presentation device 1904 may be configured for presenting the one or more statements and the at least one second information to the user. Further, the communication device 1802 may be configured for receiving a second response of the user associated with the one or more statements from the at least one user device 1902. Further, the second response may include a selection of at least one of the one or more statements and a second reaction of the user associated with the selection of the at least one of the one or more statements. Further, the processing device 1804 may be configured for analyzing the second response and the at least one second instruction using the at least one algorithm. Further, the determining of the psychological state of the user may be based on the analyzing of the second response and the at least one second instruction.

Further, in some embodiments, the processing device 1804 may be configured for generating the one or more statements using at least one first algorithm based on the analyzing of the first response and the at least one first instruction. Further, the one or more statements corresponds to the selection of the at least one of the plurality of colors.

Further, in some embodiments, the at least one user device 1902 may include at least one first sensor 1906 (as shown in FIG. 19). Further, the at least one first sensor 1906 may be configured for generating the second reaction of the user based on detecting at least one of a physiological state, a physical state, and an emotional state of the user during viewing the one or more statements.

Further, in some embodiments, the communication device 1802 may be configured for transmitting a plurality of body areas and at least one third instruction to the at least one user device 1902. Further, the at least one presentation device 1904 may be configured for presenting the plurality of body areas and the at least one third instruction. Further, the communication device 1802 may be configured for receiving a third response of the user associated with the plurality of body areas from the at least one user device 1902. Further, the third response may include a selection of at least one of the plurality of body areas and a third reaction associated with the selection of the at least one of the plurality of body areas. Further, the processing device 1804 may be configured for analyzing the third response and the at least one third instruction using the at least one algorithm. Further, the identifying of the at least one product may be based on the analyzing of the third response and the at least one third instruction.

Further, in some embodiments, the storage device 1806 may be configured for retrieving at least one user information associated with the user. Further, the processing device 1804 may be configured for analyzing the at least one user information. Further, the processing device 1804 may be configured for determining a pattern of apparel worn by the user on the plurality of body parts based on the analyzing of the at least one user information. Further, the at least one product may include at least one apparel. Further, the identifying of the at least one product may include identifying the at least one apparel of the at least one color for the user based on the determining of the pattern of apparel worn by the user.

Further, in some embodiments, the at least one user device 1902 may include at least one sensor 1908 (as shown in FIG. 19). Further, the at least one sensor 1908 may be configured for generating the first reaction of the user based on detecting at least one of a physiological state, a physical state, and an emotional state of the user during viewing the plurality of colors.

Further, in some embodiments, the at least one user device 1902 may include at least one input device 1910 (as shown in FIG. 19). Further, the at least one input device 1910 may be configured for generating the selection of the at least one of the plurality of colors based on receiving at least one user input action.

Further, in some embodiments, the storage device 1806 may be configured for retrieving one or more historical psychological states of the user associated with one or more historical selections of the at least one of the plurality of colors and the one or more historical selections of the at least one of the plurality of colors. Further, the processing device 1804 may be configured for analyzing the one or more historical psychological states and the one or more historical selections of the at least one of the plurality of colors using at least one machine learning model. Further, the at least one machine learning model may be trained in pattern detection. Further, the determining of the psychological state of the user may be based on the analyzing of the one or more historical psychological states and the one or more historical selections of the at least one of the plurality of colors.

Further, in some embodiments, the processing device 1804 may be configured for determining a plurality of first colors and at least one fourth instruction based on the analyzing of the first response and the at least one first instruction of the user. Further, the plurality of first colors does not comprise the selection of the at least one of the plurality of colors. Further, the processing device 1804 may be configured for analyzing a fourth response and the at least one fourth instruction of the user using the at least one algorithm. Further, the determining of the psychological state of the user may be based on the analyzing of the fourth response and the at least one fourth instruction. Further, the communication device 1802 may be configured for transmitting the plurality of first colors and the at least one fourth instruction to the at least one user device 1902. Further, the at least one presentation device 1904 may be configured for presenting the plurality of first colors and the at least one fourth instruction to the user. Further, the communication device 1802 may be configured for receiving the fourth response of the user associated with the plurality of first colors from the at least one user device 1902. Further, the fourth response may include a selection of at least one of the plurality of first colors and a fourth reaction of the user associated with the selection of the at least one of the plurality of first colors.

FIG. 19 is a block diagram of the system 1800 to facilitate self-analysis through data based on inputs associated with psychological states of a user, in accordance with some embodiments.

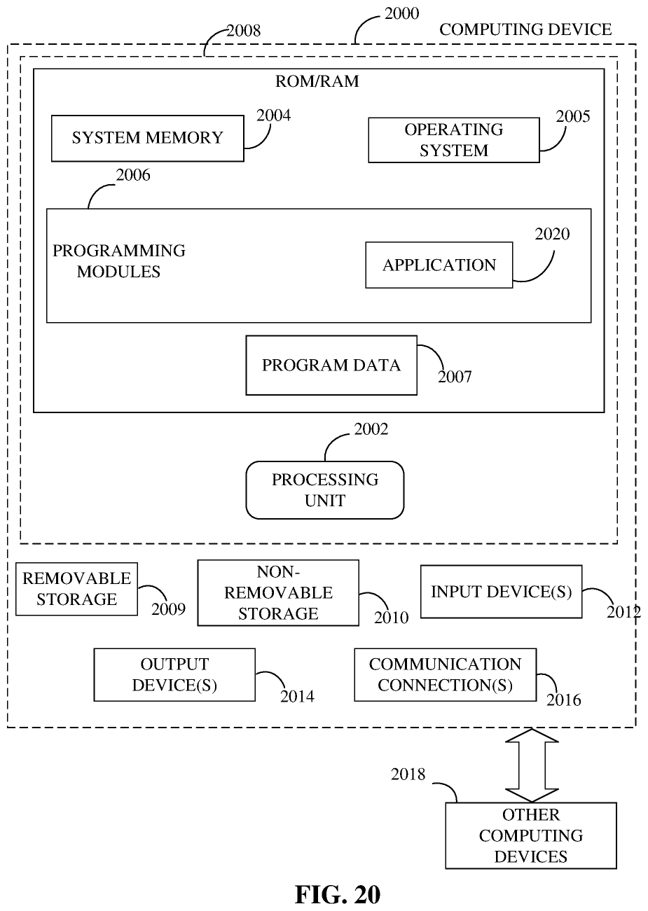

With reference to FIG. 20, a system consistent with an embodiment of the disclosure may include a computing device or cloud service, such as computing device 2000. In a basic configuration, computing device 2000 may include at least one processing unit 2002 and a system memory 2004. Depending on the configuration and type of computing device, system memory 2004 may comprise, but is not limited to, volatile (e.g. random-access memory (RAM)), non-volatile (e.g. read-only memory (ROM)), flash memory, or any combination. System memory 2004 may include operating system 2005, one or more programming modules 2006, and may include a program data 2007. Operating system 2005, for example, may be suitable for controlling computing device 2000's operation. In one embodiment, programming modules 2006 may include image-processing module, machine learning module. Furthermore, embodiments of the disclosure may be practiced in conjunction with a graphics library, other operating systems, or any other application program and is not limited to any particular application or system. This basic configuration is illustrated in FIG. 20 by those components within a dashed line 2008.

Computing device 2000 may have additional features or functionality. For example, computing device 2000 may also include additional data storage devices (removable and/or non-removable) such as, for example, magnetic disks, optical disks, or tape. Such additional storage is illustrated in FIG. 20 by a removable storage 2009 and a non-removable storage 2010. Computer storage media may include volatile and non-volatile, removable and non-removable media implemented in any method or technology for storage of information, such as computer-readable instructions, data structures, program modules, or other data. System memory 2004, removable storage 2009, and non-removable storage 2010 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 2000. Any such computer storage media may be part of device 2000. Computing device 2000 may also have input device(s) 2012 such as a keyboard, a mouse, a pen, a sound input device, a touch input device, a location sensor, a camera, a biometric sensor, etc. Output device(s) 2014 such as a display, speakers, a printer, etc. may also be included. The aforementioned devices are examples and others may be used.

Computing device 2000 may also contain a communication connection 2016 that may allow device 2000 to communicate with other computing devices 2018, such as over a network in a distributed computing environment, for example, an intranet or the Internet. Communication connection 2016 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 2004, including operating system 2005. While executing on processing unit 2002, programming modules 2006 (e.g., application 2020) may perform processes including, for example, one or more stages of methods, algorithms, systems, applications, servers, databases as described above. The aforementioned process is an example, and processing unit 2002 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readablemedium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

Although the present disclosure has been explained in relation to its preferred embodiment, it is to be understood that many other possible modifications and variations can be made without departing from the spirit and scope of the disclosure.

The following is claimed:

1. A method to facilitate self-analysis through data based on inputs associated with psychological states of a user, the method comprising:
receiving, using a communication device, a request for maintaining a mental health of the user from at least one user device associated with the user;
transmitting, using the communication device, a plurality of colors and at least one first instruction to the at least one user device based on the request, wherein the at least one user device comprises at least one presentation device, wherein the at least one presentation device is configured for presenting the at least one instruction and the plurality of colors to the user;
receiving, using the communication device, a first response of the user associated with the plurality of colors from the at least one user device, wherein the first response comprises a selection of at least one of the plurality of colors and a first reaction of the user associated with the selection of the at least one of the plurality of colors;
analyzing, using a processing device, the first response and the at least one first instruction of the user using at least one algorithm;
determining, using the processing device, a psychological state of the user associated with the selection of the at least one of the plurality of colors based on the analyzing of the first response and the at least one first instruction;
identifying, using the processing device, at least one color for the maintaining of the mental health of the user based on the determining;
identifying, using the processing device, at least one product of the at least one color for the user based on the identifying of the at least one color;
generating, using the processing device, a notification of the at least one product based on the identifying of the at least one product;
transmitting, using the communication device, the notification to the at least one user device; and
storing, using a storage device, the psychological state of the user associated with the selection of the at least one of the plurality of colors and the selection of the at least one of the list of colors.

2. The method of claim 1 further comprising:
transmitting, using the communication device, one or more statements and at least one second instruction to the at least one user device, wherein the at least one presentation device is further configured for presenting the one or more statements and the at least one second information to the user;
receiving, using the communication device, a second response of the user associated with the one or more statements from the at least one user device, wherein the second response comprises a selection of at least one of the one or more statements and a second reaction of the user associated with the selection of the at least one of the one or more statements; and
analyzing, using the processing device, the second response and the at least one second instruction using the at least one algorithm, wherein the determining of the psychological state of the user is further based on the analyzing of the second response and the at least one second instruction.

3. The method of claim 2 further comprising generating, using the processing device, the one or more statements using at least one first algorithm based on the analyzing of the first response and the at least one first instruction, wherein the one or more statements corresponds to the selection of the at least one of the plurality of colors.

4. The method of claim 2, wherein the at least one user device comprises at least one first sensor, wherein the at least one first sensor is configured for generating the second reaction of the user based on detecting at least one of a physiological state, a physical state, and an emotional state of the user during viewing the one or more statements.

5. The method of claim 1 further comprising:
transmitting, using the communication device, a plurality of body areas and at least one third instruction to the at least one user device, wherein the at least one presentation device is further configured for presenting the plurality of body areas and the at least one third instruction;
receiving, using the communication device, a third response of the user associated with the plurality of body areas from the at least one user device, wherein the third response comprises a selection of at least one of the plurality of body areas and a third reaction associated with the selection of the at least one of the plurality of body areas; and
analyzing, using the processing device, the third response and the at least one third instruction using the at least one algorithm, wherein the identifying of the at least one product is further based on the analyzing of the third response and the at least one third instruction.

6. The method of claim 5 further comprising:
retrieving, using the storage device, at least one user information associated with the user;
analyzing, using the processing device, the at least one user information; and
determining, using the processing device, a pattern of apparel worn by the user on the plurality of body parts based on the analyzing of the at least one user information, wherein the at least one product comprises at least one apparel, wherein the identifying of the at least one product comprises identifying the at least one apparel of the at least one color for the user based on the determining of the pattern of apparel worn by the user.

7. The method of claim 1, wherein the at least one user device comprises at least one sensor, wherein the at least one sensor is configured for generating the first reaction of the user based on detecting at least one of a physiological state, a physical state, and an emotional state of the user during viewing the plurality of colors.

8. The method of claim 1, wherein the at least one user device comprises at least one input device, wherein the at least one input device is configured for generating the selection of the at least one of the plurality of colors based on receiving at least one user input action.

9. The method of claim 1 further comprising:
retrieving, using the storage device, one or more historical psychological states of the user associated with one or more historical selections of the at least one of the plurality of colors and the one or more historical selections of the at least one of the plurality of colors; and
analyzing, using the processing device, the one or more historical psychological states and the one or more historical selections of the at least one of the plurality of colors using at least one machine learning model, wherein the at least one machine learning model is trained in pattern detection, wherein the determining of the psychological state of the user is further based on the analyzing of the one or more historical psychological states and the one or more historical selections of the at least one of the plurality of colors.

10. The method of claim 1 further comprising:
determining, using the processing device, a plurality of first colors and at least one fourth instruction based on the analyzing of the first response and the at least one first instruction of the user, wherein the plurality of first colors does not comprise the selection of the at least one of the plurality of colors;
transmitting, using the communication device, the plurality of first colors and the at least one fourth instruction to the at least one user device, wherein the at least one presentation device is further configured for presenting the plurality of first colors and the at least one fourth instruction to the user;
receiving, using the communication device, a fourth response of the user associated with the plurality of first colors from the at least one user device, wherein the fourth response comprises a selection of at least one of the plurality of first colors and a fourth reaction of the user associated with the selection of the at least one of the plurality of first colors; and
analyzing, using the processing device, the fourth response and the at least one fourth instruction of the user using the at least one algorithm, wherein the determining of the psychological state of the user is further based on the analyzing of the fourth response and the at least one fourth instruction.

11. A system to facilitate self-analysis through data based on inputs associated with psychological states of a user, the system comprising:
a communication device configured for:
receiving a request for maintaining a mental health of the user from at least one user device associated with the user;
transmitting a plurality of colors and at least one first instruction to the at least one user device based on the request, wherein the at least one user device comprises at least one presentation device, wherein the at least one presentation device is configured for presenting the at least one instruction and the plurality of colors to the user;
receiving a first response of the user associated with the plurality of colors from the at least one user device, wherein the first response comprises a selection of at least one of the plurality of colors and a first reaction of the user associated with the selection of the at least one of the plurality of colors; and
transmitting a notification to the at least one user device;
a processing device communicatively coupled with the communication device, wherein the processing device is configured for:
analyzing the first response and the at least one first instruction of the user using at least one algorithm;
determining a psychological state of the user associated with the selection of the at least one of the plurality of colors based on the analyzing of the first response and the at least one first instruction;
identifying at least one color for the maintaining of the mental health of the user based on the determining;
identifying at least one product of the at least one color for the user based on the identifying of the at least one color; and
generating the notification of the at least one product based on the identifying of the at least one product; and
a storage device communicatively coupled with the processing device, wherein the storage device is configured for storing the psychological state of the user associated with the selection of the at least one of the plurality of colors and the selection of the at least one of the list of colors.

12. The system of claim 11, wherein the communication device is further configured for:
transmitting one or more statements and at least one second instruction to the at least one user device, wherein the at least one presentation device is further configured for presenting the one or more statements and the at least one second information to the user; and
receiving a second response of the user associated with the one or more statements from the at least one user device, wherein the second response comprises a selection of at least one of the one or more statements and a second reaction of the user associated with the selection of the at least one of the one or more statements, wherein the processing device is further configured for analyzing the second response and the at least one second instruction using the at least one algorithm, wherein the determining of the psychological state of the user is further based on the analyzing of the second response and the at least one second instruction.

13. The system of claim 12, wherein the processing device is further configured for generating the one or more statements using at least one first algorithm based on the analyzing of the first response and the at least one first instruction, wherein the one or more statements corresponds to the selection of the at least one of the plurality of colors.

14. The system of claim 12, wherein the at least one user device comprises at least one first sensor, wherein the at least one first sensor is configured for generating the second reaction of the user based on detecting at least one of a physiological state, a physical state, and an emotional state of the user during viewing the one or more statements.

15. The system of claim 11, wherein the communication device is further configured for:
transmitting a plurality of body areas and at least one third instruction to the at least one user device, wherein the at least one presentation device is further configured for presenting the plurality of body areas and the at least one third instruction; and
receiving a third response of the user associated with the plurality of body areas from the at least one user device, wherein the third response comprises a selection of at least one of the plurality of body areas and a third reaction associated with the selection of the at least one of the plurality of body areas, wherein the processing device is further configured for analyzing the third response and the at least one third instruction using the at least one algorithm, wherein the identifying of the at least one product is further based on the analyzing of the third response and the at least one third instruction.

16. The system of claim 15, wherein the storage device is further configured for retrieving at least one user information associated with the user, wherein the processing device is further configured for:
analyzing the at least one user information; and
determining a pattern of apparel worn by the user on the plurality of body parts based on the analyzing of the at least one user information, wherein the at least one product comprises at least one apparel, wherein the identifying of the at least one product comprises identifying the at least one apparel of the at least one color for the user based on the determining of the pattern of apparel worn by the user.

17. The system of claim 11, wherein the at least one user device comprises at least one sensor, wherein the at least one sensor is configured for generating the first reaction of the user based on detecting at least one of a physiological state, a physical state, and an emotional state of the user during viewing the plurality of colors.

18. The system of claim 11, wherein the at least one user device comprises at least one input device, wherein the at least one input device is configured for generating the selection of the at least one of the plurality of colors based on receiving at least one user input action.

19. The system of claim 11, wherein the storage device is further configured for retrieving one or more historical psychological states of the user associated with one or more historical selections of the at least one of the plurality of colors and the one or more historical selections of the at least one of the plurality of colors, wherein the processing device is further configured for analyzing the one or more historical psychological states and the one or more historical selections of the at least one of the plurality of colors using at least one machine learning model, wherein the at least one machine learning model is trained in pattern detection, wherein the determining of the psychological state of the user is further based on the analyzing of the one or more historical psychological states and the one or more historical selections of the at least one of the plurality of colors.

20. The system of claim 11, wherein the processing device is further configured for:
determining a plurality of first colors and at least one fourth instruction based on the analyzing of the first response and the at least one first instruction of the user, wherein the plurality of first colors does not comprise the selection of the at least one of the plurality of colors; and
analyzing a fourth response and the at least one fourth instruction of the user using the at least one algorithm, wherein the determining of the psychological state of the user is further based on the analyzing of the fourth response and the at least one fourth instruction, wherein the communication device is further configured for:
transmitting the plurality of first colors and the at least one fourth instruction to the at least one user device, wherein the at least one presentation device is further configured for presenting the plurality of first colors and the at least one fourth instruction to the user; and
receiving the fourth response of the user associated with the plurality of first colors from the at least one user device, wherein the fourth response comprises a selection of at least one of the plurality of first colors and a fourth reaction of the user associated with the selection of the at least one of the plurality of first colors.
